(19) United States
(12) Patent Application Publication
(10) Pub. No.: US 2025/0042840 A1
(43) Pub. Date: Feb. 6, 2025

(54) PROCESS FOR PREPARING TERTIARY AMINES

(71) Applicants: SPECIALTY OPERATIONS FRANCE, Lyon (FR); LE CENTRE NATIONAL DE LA RECHERCHE SCIENTIFIQUE, Paris Cedex 16 (FR); UNIVERSITE DE POITIERS, Poitiers (FR)

(72) Inventors: Raphael Johannes Wischert, Shanghai (CN); Marc Pera Titus, Paris Cedex 16 (FR); Francois Jerome, 3 Rue Michel Ange (FR); Gongming Peng, Shanghai (CN); Stephane Streiff, Aubervilliers (FR)

(73) Assignees: SPECIALTY OPERATIONS FRANCE, Lyon (FR); LE CENTRE NATIONAL DE LA RECHERCHE SCIENTIFIQUE, Paris Cedex 16 (FR); UNIVERSITE DE POITIERS, Poitiers (FR)

(21) Appl. No.: 18/717,923

(22) PCT Filed: Dec. 21, 2021
(86) PCT No.: PCT/CN2021/139864

§ 371 Date: Jun. 7, 2024

(51) Int. Cl. C07C 209/78; C07C 209/60

(52) U.S. Cl. CPC C07C 209/78; C07C 209/60; C07C 2601/16; C07C 2601/18; C07C 2602/42

(57) ABSTRACT

Provided is a simple and environmentally friendly process for preparing tertiary amines by the aminomethylation of alkenes. This process features relatively mild reaction conditions and no metal catalyst.

### TECHNICAL FIELD

The present invention relates to a process for preparing tertiary amines.

### BACKGROUND

The following discussion of the prior art is provided to place the invention in an appropriate technical context and enable the advantages of it to be more fully understood. It should be appreciated, however, that any discussion of the prior art throughout the specification should not be considered as an express or implied admission that such prior art is widely known or forms part of common general knowledge in the field.

Tertiary alkylamines are widely used in synthetic chemistry and feedstock-chemical processing alike. The application segments for tertiary alkylamines with the largest growth are such as surfactants, followed by biocides, flotation agents, corrosion inhibitors, drilling materials and emulsifiers. The demand for tertiary alkylamines is also rapidly increasing in other end-use industries such as personal care, petroleum extraction, water treatment, plastics, pharmaceuticals, and textile and fiber industries.

At industrial scale, tertiary alkylamines are mostly produced from alkyl alcohols and secondary amines through a tandem dehydrogenation-reductive amination reaction in the presence of a supported transition metal catalyst and hydrogen. However, this reaction remains a very challenging task. The recovery and recycling of metal complexes after the reaction is difficult or prohibitively expensive. Especially, the synthesis of linear tertiary alkylamines is an additional challenge, as metal complexes yield branched or a mixture of branched or linear tertiary amines as main products.

Alkene is cheap and widely used in the modern industry as raw materials. It is very challenge to obtain tertiary amines direct from alkene and secondary amines under mild condition.

Therefore, there is still a need in the industry and it would be desirable to find alternative, improved, mild-condition and metal-free process for preparing tertiary amines. Such process steps are relatively simple, which highly improves economic efficiency.

### SUMMARY OF THE INVENTION

The aim of the present invention is then to provide a process preparing tertiary amines, which features a simple and environmental-friendly system and relatively mild reaction conditions, and no transition metal catalysts employed with apparently improved economic benefit.

Upon diligent research, the applicants have discovered surprisedly that such an aim can be achieved by selecting a specific alkene and secondary amine in the presence of formica acid and a reducing agent.

Thus, the present invention is directed to a process for preparing a tertiary amine by a reaction of an alkene comprising a liner alkene represented by formula (I) or a cycloalkene and a secondary amine represented by formula (II) in the presence of formaldehyde and a reducing agent,

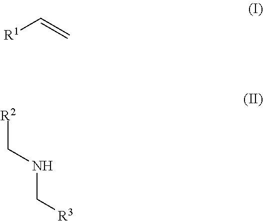

wherein:
R¹, R², and R³ are same or different and each independently H or a hydrocarbon radical which is optionally interrupted by one or more heteroatoms and/or heteroatom(s) containing groups and/or which is optionally substituted with one or more functional groups.

With the process according to the present invention, it is possible to prepare tertiary amine(s) under mild reaction conditions, i.e. at a temperature lower than 80° C.

In addition, the tertiary amine can be prepared without using a basic compound and a metal catalyst. Thus, this process is more simple and environmental-friendly.

Furthermore, the starting reactants, especially the starting secondary amines can be used directly without pre-treatment, such as prior conversion into salts.

Other subjects and characteristics, aspects and advantages of the present invention will emerge even more clearly on reading the detailed description and the examples that follow.

### Definitions

Throughout the description, including the claims, the term “comprising a” should be understood as being synonymous with the term “comprising at least a”, unless otherwise specified, and “between” should be understood as being inclusive of the limits.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

It is specified that, in the continuation of the description, unless otherwise indicated, the values at the limits are included in the ranges of values which are given.

Ratios, concentrations, amounts, and other numerical data may be presented herein in a range format. It is to be understood that such a range format is used merely for convenience and brevity and should be interpreted flexibly to include not only the numerical values explicitly recited as the limits of the range, but also all the individual numerical values or sub-ranges encompassed within that range as if each numerical value or sub-range is explicitly recited.

Unless defined in other ways, all technical and scientific terms used herein have the same meaning as commonly understood by those skilled in the field to which this specification relates.

### DETAILS OF THE INVENTION

The present invention provides a process for preparing a tertiary amine by a reaction of an alkene comprising a liner alkene represented by formula (I) or a cycloalkene and a secondary amine represented by formula (II) in the presence of formaldehyde and a reducing agent,

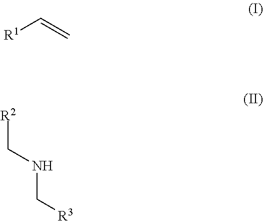

wherein:
R¹, R², and R³ are same or different and each independently H or a hydrocarbon radical which is optionally interrupted by one or more heteroatoms and/or heteroatom(s) containing groups and/or which is optionally substituted with one or more functional groups.

In some embodiments, R¹ is an alkyl having carbon atoms from 1 to 24, preferably from 1 to 18, even more preferably from 1 to 16 (such as methyl, ethyl, propyl, butyl, pentyl, hexyl, heptyl, octyl, nonyl or decyl), an aryl or heteroaryl, optionally substituted or optionally further substituted with one or more functional groups.

In some embodiments, R¹ is H.

As previously expressed, R² and R³ are same or different and each independently a hydrocarbon radical which is optionally interrupted by one or more heteroatoms and/or heteroatom(s) containing groups and/or which is optionally substituted with one or more functional groups.

In some embodiments, R² and R³ are different and each independently a C₁-C₂₄ alkyl, preferably a C₁-C₁₆ alkyl, more preferably C₁-C₁₂ alkyl, even more preferably C₁-C₁₀ alkyl, such as methyl, ethyl, propyl, butyl, pentyl, hexyl, heptyl, octyl, nonyl or decyl, optionally substituted or optionally further substituted with one or more functional groups.

In some embodiments, R² and R³ are same, such as a C₁-C₂₄ alkyl, preferably a C₁-C₁₆ alkyl, more preferably C₁-C₁₂ alkyl, even more preferably C₁-C₁₀ alkyl, such as methyl, ethyl, propyl, butyl, pentyl, hexyl, heptyl, octyl, nonyl or decyl, optionally substituted or optionally further substituted with one or more functional groups.

In some embodiments, R² and R³ are same or different and each independently aryl or heteroaryl, optionally substituted or optionally further substituted with one or more functional groups, such as benzyl group, phenethyl group and etc.

In some embodiments, the secondary amines can be selected from symmetrical amines such as diethylamine, dibutylamine, dipropyl, dibenzylamines or the mixture thereof.

In some embodiments, the secondary amines can be selected from unsymmetrical secondary amines comprising N-ethyl, N-propyl, N-pentyl- and N-octylmethylamine, a mixture of N-dimethylated and N-monomethylated amines, such as N-methyl-ethylamine, N-methyl-propylamine, N-methyl-propylamine, N-methyl-octylamine or mixture thereof.

In some embodiments, the alkene represented by general formula (I) can be styrene or styrene substituted with alkyl, phenyl, halo or alkoxy. Said alkyl can be a C₁-C₁₆ straight or branched chain alkyl. Preferably, C₁-C₆ straight chain alkyl can be selected from the group consisting of methyl, ethyl, 1-propyl, n-butyl and n-pentyl. Preferably, C₁-C₆ branched chain alkyl can be isopropyl or isobutyl. Said alkoxy preferably can be a C₁-C₆ alkoxy and more preferably methoxy or ethoxy. Said halo can be F, Cl, Br or I.

In some embodiments, the liner alkene represented by general formula (I) is selected from 1-pentene, 1-hexene, 1-heptene, 1-octene, 1-nonene, 1-decene, 1-undecene, 1-dodecene, 1-tridecene, or the mixture thereof.

In some embodiments, the liner alkene represented by general formula (I) is selected from 1-fluoro-4-vinylbenzene, 1-chloro-4-vinylbenzene, 1-chloro-2-vinylbenzene, 1-chloro-3-vinylbenzene, 1-bromo-3-vinylbenzene, 1-methyl-2-vinylbenzene, 4-vinyl-1,1′-biphenyl, 1-(tert-butyl)-4-vinylbenzene, or the mixture thereof.

Cyclic alkene or cycloalkene are hydrocarbons containing a ring of carbon atoms and one or more double bonds in the cycle that do not form an aromatic ring, such as five-membered ring, six-membered ring, seven-membered ring, eight-membered ring. It can be monocyclic, bicyclic or polycyclic alkene, which can be substituted, optionally further substituted, with one or more functional groups.

In some embodiments, the cyclioalkene is selected from cyclohexene, cyclooctene or norbornene, which is optionally substituted or optionally further substituted with one or more functional group.

The reducing agent as used herein is to prevent the reduction of alkene and provide hydride donor to prevent the formation of quaternary amines.

In some embodiments, the reducing agent is selected from formic acid, sodium cyanoborohydride, sodium borohydride, sodium tetrahydroborate, potassium borohydride, potassium tetrahydroborate, preferably selected from formic acid, sodium cyanoborohydride or sodium borohydride, more preferably selected from formic acid or sodium cyanoborohydride, most preferably selected from formic acid.

In some embodiments, according to the process of the present invention, at least one solvent is further employed and can be a compound having the following general formula (III).

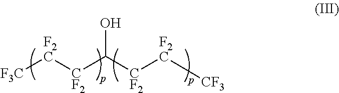

wherein p is an integer from 0 to 10.

Non limitative examples of the compound having the general formula (III) is hexafluoroisopropanol (HFIP).

The term “alkyl” as used herein refers to a linear, branched, or cyclic saturated hydrocarbon group, which typically (though not necessarily) contains 1 to about 24 carbon atoms, or 1 to about 18 carbon atoms, or 1 to about 16 carbon atoms, or 1 to about 16 carbon atoms. The alkyl can be substituted alkyl, optionally further substituted, with one or more functional groups. Certain embodiments provide that the alkyl group is methyl, ethyl, n-propyl, isopropyl, n-butyl, isobutyl, tert-butyl, octyl, decyl, etc., and cycloalkyl groups such as cyclopentyl group, cyclohexyl, etc. Generally, although it is not necessary again, the alkyl group here contains 1 to about 14 carbon atoms. The term “cycloalkyl” means a cyclic alkyl group, typically having 4 to 8, preferably 5 to 8 carbon atom. The term “substituted alkyl” refers to an alkyl group substituted with one or more substituent groups, and includes “heteroatom-containing alkyl” and “heteroalkyl”, these terms refer to where a heteroatom replaces at least one carbon atom of the alkyl group. If not otherwise specified, the term “alkyl” include linear, branched, cyclic, unsubstituted, substituted, and/or heteroatom-containing alkyl, respectively.

As used herein, the term “aryl” means a monocyclic or bicyclic aromatic hydrocarbon radical of 6 to 10 ring atoms which is optionally substituted independently with one to four substituents, preferably one, two, or three substituents selected from alkyl, alkenyl, alkynyl, aryl, halo, nitro, cyano, hydroxy, alkoxy, amino, mono-alkylamino, di-alkylamino and heteroalkyl.

As used herein, the term “heteroaryl” means a monocyclic or bicyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to four substituents, preferably one or two substituents, selected from alkyl, aryl, halo, nitro, cyano, hydroxy, alkoxy, amino, acylamino, mono-alkylamino, di-alkylamino, heteroalkyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl.

“Functional” as in “functional groups” means that in the alkyl, aryl, alkene, cycloalkene, amines or other moiety or group, it is at least one hydrogen atom bound on a carbon (or other) atom is replaced with one or more functional groups (such as those described here and above). The term “functional group” is meant to include any functional species suitable for the use described herein. Specifically, as used herein, the functional group will have to have the ability to react with or bind to the corresponding functional group on the surface of the substrate.

The terms “cyclic” and “cyclo” refer to alicyclic or aromatic groups, which may or may not be substituted and or heteroatom-containing, and which may be monocyclic, bicyclic, or polycyclic. The term “alicyclic” is used in the conventional sense to refer to an aliphatic cyclic moiety, as opposed to an aromatic cyclic moiety, and may be monocyclic, bicyclic, or polycyclic.

In addition, if the specific group permits, the above-mentioned functional group may be further substituted with one or more additional functional groups (such as those specifically listed above). Similarly, the aforementioned groups may be further substituted with one or more functional groups (such as those specifically enumerated).

As used herein, the indication that a group can be “optionally substituted” or “optionally further substituted” generally means that such a group unless explicitly or further defined by the context of such reference The group can be substituted by one or more inorganic or organic substituents (such as alkyl, alkenyl, aryl, aralkyl, alkaryl, heteroatom, or heterocyclic group), or can be coordinated by one or more functional groups to metal ions (such as hydroxyl, carbonyl, carboxyl, amino, imino, amido, phosphonic acid, sulfonic acid, or arsenate, or inorganic and organic esters thereof, such as sulfate or phosphate, or salts thereof) replace.

According to the process of the present invention, formaldehyde can be introduced in the form of an aqueous solution. The concentration of formaldehyde in the aqueous solution can be from 35% to 55% and preferably from 35% to 40%. In a preferred embodiment, the aqueous solution of formaldehyde can be formalin.

Advantageously, the molar ratio of the alkene to the formaldehyde and the secondary amine is from 1:1.2:1.2 to 1:10:10, preferably 1:2:2 to 1:9:9, more preferably 1:4:4 to 1:8:8.

Advantageously, the molar ratio of the alkene to the reducing agent is from 1:1.2 to 1:16, preferably 1:2 to 1:14, more preferably 1:3 to 1:10, even more preferably 1:4 to 1:8.

According to the process of the present invention, the reaction temperature can be lower than 80° C., preferably lower than 60° C. and most preferably lower than 55° C. Advantageously, the reaction temperature is in the range of 20° C. to 60° C., preferably in the range of 25° C. to 55° C., even more preferably in the range of 30° C. to 50° C.

According to the process of the present invention, the reaction time is not particularly limited. The preferred reaction time can be from 10 to 120 hours, such as 16, 17, 18, 96, 110 hours.

The process of the present invention may comprise following steps:
a) mixing formaldehyde, an alkene comprising a liner alkene represented by formula (I) or a cycloalkene and a secondary amine represented by general formula (II);
b) adding a reducing agent, optional a solvent represented by the formula (III) to the mixture obtained in step a) to obtain a reaction mixture;
c) maintaining the reaction mixture obtained in step b) under proper reaction temperature and proper reaction time to obtain at least one tertiary amine.

The alkene, the secondary amine represented by general formula (II), the reducing agent and the solvent represented by the formula (III), the reaction temperature and the reaction time are as defined above.

Advantageously, the process of the present invention can be a one-pot reaction as there is no need to hydrolysis of a tertiary amine salt to obtain the tertiary amine.

An aspect of the present invention also provides a composition comprising:
(i) formaldehyde,
(ii) at least one alkene comprising a liner alkene represented by formula (I) or a cycloalkene,
(iii) at least one secondary amine represented by general formula (II),
(iv) a reducing agent, and
(v) optionally a solvent represented by the formula (III).

The alkene comprising a liner alkene represented by formula (I) or a cycloalkene, the secondary amine represented by general formula (II), the reducing agent and the solvent represented by the formula (III) are as defined above.

The following examples are included to illustrate embodiments of the invention. Needless to say, the invention is not limited to describe examples.

### Experimental Part

### Materials

Dimethylamine (DMA) aqueous (38 wt % aqueous, reagent grade), cas: 100-42-5, Merck
Formalin (37 wt % aqueous, reagent grade), cas: 124-40-3, Merck
HCOOH (reagent grade), cas: 64-18-6, Aladin
Hexafluoroisopropanol (HFIP) (reagent grade), cas: 920-66-1, Aladin
1-octene (reagent grade), cas: 111-66-0, Aladin
1-Pentene (reagent grade), cas: 109-67-1, Aladin
1-Hexene (reagent grade), cas: 592-41-6, Aladin
1-Heptene (reagent grade), cas: 592-76-7, Aladin
1-Decene (reagent grade), cas: 872-05-9, Aladin
1-Undecene (reagent grade), cas: 821-95-4, Aladin
1-Dodecene (reagent grade), cas: 112-41-4, Aladin
1-Tridecene (reagent grade), cas: 2437-56-1, Aladin
1-Cyclooctene (reagent grade), cas: 931-87-3, Aladin
Styrene (reagent grade), cas: 100-42-5, Aladin
Norbornene (reagent grade), cas: 498-66-8, Aladin
Diethylamine (reagent grade), cas: 109-89-7, Aladin
Dipropylamine (reagent grade), cas: 142-84-7, Aladin
Dibutylamine (reagent grade), cas: 111-92-2, Aladin
Dibenzylamine (reagent grade), cas: 103-49-1, Aladin
N-methyl-ethylamine (reagent grade), cas: 624-78-2, Aladin
N-methyl-propylamine (reagent grade), cas: 627-35-0, Aladin
N-methyl-pentylamine (reagent grade), cas: 25419-6-1, Aladin
N-methyl-octylamine (reagent grade), cas: 2439-54-5, Aladin

### Analytical Method

After completion of the reaction, the mixture was filtrated and analyzed using an Agilent 7890 GC equipped with an HP-5 capillary column bearing 5 wt % phenyl groups (length 30 m; inner diameter 0.25 mm). Analytical methods were adjusted for the different mixtures depending on the boiling point and polarity of the reagents and products. In all the methods, the injector temperature was set at 250° C., the detector temperature was 300° C. and the sample injection volume was 1 μL. The calibration of the gas chromatography was performed using dodecanol as an internal standard.

### NMR Spectroscopy

The product was analyzed by NMR on a Bruker Avance III 300 MHz spectrometer operating at 300 MHz resonance frequencies, equipped with a BBO probe.

### Example 1: Preparation of Tertiary Amines from 1-Octene/Formaldehyde/DMA/HCOOH

A round-bottom flask was charged with HCHO (4 mmol, 37 wt %, formalin), DMA (4 mmol, 38 wt % in water) and HFIP (10 mL). Then, 1-octene (112 mg, 1 mmol, 1 equiv.) and HCOOH (184 mg, 4 mmol) was added at room temperature and the reaction mixture was stirred at 30° C. After 96 h, the mixture was filtrated and analyzed using an Agilent 7890 GC equipped with an HP-5 capillary column bearing 5 wt % phenyl groups (length 30 m; inner diameter 0.25 mm).

TABLE 1

| | Reactant | Product | Selectivity | Yield |
|---|---|---|---|---|
| EX | (alkene) | (tertiary amine) | (%) | (%) |
| 1 | 1-octene | N,N-dimethylamine | 85 | 64 |

### Examples 2-11: Preparation of Tertiary Amines from Different Alkenes

General procedure of Examples 2-11 is same as Example 1. The results are summarized in Table 2.

TABLE 2^{a}

| | Reactant | Product | Yield^{b} |
|---|---|---|---|
| EX | (alkene) | (tertiary amine) | (%) |
| 2 | 1-Pentene | N,N-dimethyl-hexylamine | (36^{c}) |
| 3 | 1-Hexene | N,N-dimethyl-heptylamine | 50 |
| 4 | 1-Heptene | N,N-dimethyl-octylamine | 51(45^{c}) |
| 5 | 1-Decene | N,N-dimethyl-undecylamine | 60 |
| 6 | 1-Undecene | N,N-dimethyl-dodecylamine | 70(56^{c}) |
| 7 | 1-Dodecene | N,N-dimethyl-tridecylamine | 68 |
| 8 | 1-Tridecene | N,N-dimethyl-tetradecylamine | 72 |
| 9 | 1-Cyclooctene | N,N-dimethyl-cyclooctane methylamine | 58 |
| 10 | Styrene | N,N-dimethyl-amphetamine | 73 |
| 11 | Norbornene | N,N-dimethyl-norbornane methylamine | 65 |

^{a}Reaction conditions: 1 mmol alkene, 4 mmol DMA, 4 mmol HCHO (formalin solution), 4 mmol HCOOH, 10 mL HFIP, 30° C.;
^{b}GC yield;
^{c}isolated yield.

### Examples 12-19: Preparation of Tertiary Amines from Different Amines

Regarding other secondary alkylamines (examples 12-20), a same experimental procedure was used as example 1, except that the temperature of the reaction was raised to 50° C., the reaction time prolong to 120 h. The results are summarized in Table 3.

TABLE 3^{a}

| | Reactant | Product | | Yield^{c} |
|---|---|---|---|---|
| EX | (amines) | (tertiary amine) | Ratio^{b} | (%) |
| 12 | diethylamine | N-methyl-N-ethyl-nonylamine | — | 36 |
| 13 | dipropylamine | N-methyl-N-propyl-nonylamine | — | 67 |
| 14 | dibutylamine | N-methyl-N-butyl-nonylamine | — | 53 |
| 15 | dibenzylamine | N-methyl-N-benzyl-nonylamine | — | 65 |
| 16 | N-methyl-ethylamine | N-methyl-N-ethyl-nonylamine: | 50:50 | 57 |
| | | N,N-dimethyl-nonylamine | | |
| 17 | N-methyl-propylamine | N-methyl-N-propyl-nonylamine: | 50:50 | 74 |
| | | N,N-dimethyl-nonylamine | | |
| 18 | N-methyl-pentylamine | N-methyl-N-pentyl-nonylamine: | 50:50 | 68 |
| | | N,N-dimethyl-nonylamine | | |
| 19 | N-methyl-octylamine | N-methyl-N-octyl-nonylamine: | 56:44 | 80 |
| | | N,N-dimethyl-nonylamine | | |

^{a}Reaction conditions: 1 mmol 1-octene, 4 mmol amine, 4 mmol HCHO (formalin solution), 4 mmol HCOOH, 10 mL HFIP, 50° C., 120 h;
^{b}ratio of former to latter amine (products);
^{c}GC yield of total amines.

1. A process for preparing a tertiary amine by a reaction of an alkene comprising a liner alkene represented by formula (I) or a cycloalkene and a secondary amine represented by formula (II) in the presence of formaldehyde and a reducing agent, 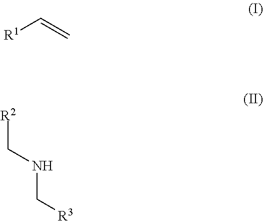
wherein:
R¹, R², and R³ are the same or different and each independently are H or a hydrocarbon radical, wherein the hydrocarbon radical is optionally interrupted by one or more heteroatoms and/or heteroatom(s) containing groups and/or which is optionally substituted with one or more functional groups.

2. The process according to claim 1, wherein R¹ is an alkyl having carbon atoms from 1 to 24, an aryl or heteroaryl, and is optionally substituted or optionally further substituted with one or more functional groups.

3. The process according to claim 1, wherein R² and R³ are the same or different and each independently comprise a C₁-C₂₄ alkyl, and are optionally substituted or optionally further substituted with one or more functional groups.

4. The process according to claim 1, wherein R² and R³ are the same or different and each independently comprise aryl or heteroaryl, and are optionally substituted or optionally further substituted with one or more functional groups.

5. The process according to claim 1, wherein the cycloalkene comprises a heteroatom in the ring.

6. The process according to claim 1, wherein the cycloalkene is selected from the group consisting of cyclohexene, cyclooctene, norbornene and mixtures thereof.

7. The process according to claim 1, wherein the liner alkene is selected from the group consisting of 1-pentene, 1-hexene, 1-heptene, 1-octene, 1-nonene, 1-decene, 1-undecene, 1-dodecene, 1-tridecene, and mixtures thereof.

8. The process according to claim 1, wherein the liner alkene is selected from the group consisting of styrene, 1-fluoro-4-vinylbenzene, 1-chloro-4-vinylbenzene, 1-chloro-2-vinylbenzene, 1-chloro-3-vinylbenzene, 1-bromo-3-vinylbenzene, 1-methyl-2-vinylbenzene, 4-vinyl-1,1′-biphenyl, 1-(tert-butyl)-4-vinylbenzene, and mixtures thereof.

9. The process according to claim 1, wherein the secondary amine is selected from symmetrical secondary amines; or unsymmetrical secondary amines.

10. The process according to claim 1, wherein the reducing agent is selected from the group consisting of formic acid, sodium cyanoborohydride, sodium borohydride, sodium tetrahydroborate, potassium borohydride, potassium tetrahydroborate, and mixtures thereof.

11. The process according to claim 10, wherein the reducing agent is selected from formic acid, sodium cyanoborohydride or sodium borohydride.

12. The process according to claim 1, wherein the reaction is in the presence of a solvent having the following general formula (III), 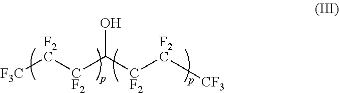
wherein p is an integer from 0 to 10.

13. The process according to claim 12, wherein the solvent is hexafluoroisopropanol.

14. The process according to claim 1, wherein a molar ratio of the alkene to the formaldehyde and the secondary amine is from 1:1.2:1.2 to 1:10:10.

15. The process according to claim 1, wherein a molar ratio of the alkene to the reducing agent is from 1:1.2 to 1:16.

16. The process according to claim 1, wherein the reaction temperature is in the range of 20° C. to 60° C.

17. The process according to claim 1, wherein the reaction time is from 10 to 120 hours.

18. The process according to claim 1, the process comprising the steps of:
a) mixing formaldehyde, and the alkene comprising a liner alkene represented by formula (I) or the cycloalkene and the secondary amine represented by general formula (II);
b) adding the reducing agent, and optionally a solvent represented by the formula (III) to the mixture obtained in step a) to obtain a reaction mixture;
c) maintaining the reaction mixture obtained in step b) under proper reaction temperature and proper reaction time to obtain at least one tertiary amine,
wherein,
the alkene, the secondary amine represented by general formula (II), and the reducing agent are as defined as claim 1,
the solvent represented by formula (III), 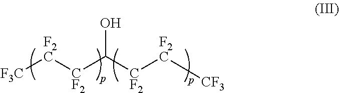
wherein p is an integer from 0 to 10,
the reaction temperature is in the range of 20° C. to 60° C., and
the reaction time is from 10 to 120 hours.

19. A composition comprising:
(i) formaldehyde,
(ii) an alkene,
(iii) a secondary amine,
(iv) a reducing agent, and
(v) optionally a solvent represented by formula (III), 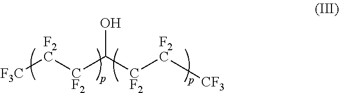
wherein p is an integer from 0 to 10, and
wherein
the alkene comprising a liner alkene represented by formula (I) or a cycloalkene, the secondary amine represented by general formula (II), the reducing agent are as defined as claim 1.

20. The process according to claim 1, wherein R1 is an alkyl having carbon atoms from 1 to 18, an aryl or heteroaryl, and is optionally substituted or optionally further substituted with one or more functional groups.
